(19) United States
(12) Patent Application Publication
(10) Pub. No.: US 2022/0031407 A1
(43) Pub. Date: Feb. 3, 2022

(54) SURGICAL ROBOTIC SYSTEMS

(71) Applicant: Covidien LP, Mansfield, MA (US)

(72) Inventors: Jaimeen Kapadia, Cambridge, MA (US); Mark Macleod, Southbury, CT (US); Colin H. Murphy, Cambridge, MA (US)

(21) Appl. No.: 17/276,531

(22) PCT Filed: Sep. 10, 2019
(86) PCT No.: PCT/US19/50277

§ 371 Date: Mar. 16, 2021

Related U.S. Application Data
(60) Provisional application No. 62/732,149, filed on Sep. 17, 2018.

(51) Int. Cl. A61B 34/30; A61B 34/00; A61B 90/00

(52) U.S. Cl. CPC A61B 34/30; A61B 2090/064; A61B 90/06; A61B 34/74

(57) ABSTRACT

A surgical robotic system includes a robotic arm including a plurality of elongate members rotationally coupled to one another, a first activation button coupled to one of the elongate members, a first force sensor coupled to one of the elongate members, and a processor. One of the elongate members is configured to have a surgical instrument attached thereto. The first activation button is configured to actuate a function, and the first force sensor is configured to sense a force associated with an actuation of the first activation button. The processor is in communication with the first force sensor and is configured to determine whether an actuation of the first activation button is intentional or accidental based on data received from the first force sensor.

### BACKGROUND

Robotic surgical systems have been used in minimally invasive medical procedures. Some robotic surgical systems included a console supporting a surgical robotic arm and a surgical instrument or at least one end effector (e.g., forceps or a grasping tool) mounted to the robotic arm. The robotic arm provided mechanical power to the surgical instrument for its operation and movement. Each robotic arm may have included an instrument drive unit operatively connected to the surgical instrument.

The instrument drive unit was typically coupled to the robotic arm via a rail. The rail allowed the instrument drive unit and the attached surgical instrument to move along an axis of the rail, providing a means for adjusting the axial position of the end effector of the surgical instrument.

### SUMMARY

In one aspect of the present disclosure, a surgical robotic system is provided and includes a robotic arm including a plurality of elongate members rotationally coupled to one another, a first activation button coupled to one of the plurality of elongate members, a first force sensor coupled to one of the elongate members, and a processor. One of the elongate members is configured to have a surgical instrument attached thereto. The first activation button is configured to actuate a function, and the first force sensor is configured to sense a force associated with an actuation of the first activation button. The processor is in communication with the first force sensor and configured to determine whether an actuation of the first activation button is intentional or accidental based on data received from the first force sensor.

In aspects, the elongate members may include a rail having the surgical instrument slidably coupled thereto. The first activation button may be disposed with the rail.

In some aspects, the surgical robotic system may further include a second activation button attached to an instrument drive unit of the surgical robotic system. The instrument drive unit may be configured to drive an operation of the surgical instrument.

In further aspects, the surgical robotic system may further include a second force sensor configured to sense a force associated with an actuation of the second activation button.

In other aspects, the elongate members may include a first elongate member having a first end and a second end, a second elongate member having a first end rotatably connected to the second end of the first elongate member, a third elongate member having a first end rotatably connected to a second end of the second elongate member, and a rail rotatably coupled to a second end of the third elongate member. The rail may have the surgical instrument slidably coupled thereto, and the first activation button may be disposed with the rail.

In aspects, the surgical robotic system may further include a base having the first end of the first elongate member rotatably coupled thereto.

In some aspects, the surgical robotic system may further include a second activation button attached to the surgical instrument, an instrument drive unit, or the robotic arm, and a second force sensor disposed with the base. The second activation button may be configured to actuate a function, and the second force sensor may be configured to sense a force associated with an actuation of the second activation button.

In further aspects, the first elongate member may be configured to rotate relative to the base about a first pivot axis, and the second elongate member may be configured to rotate relative to the first elongate member about a second pivot axis, extending perpendicularly relative to the first pivot axis.

In other aspects, the surgical instrument may be configured to move along a longitudinal axis defined by the rail.

In aspects, the processor may be configured to determine whether an actuation of the first activation button is intentional or accidental by determining if the force sensed by the first force sensor is below a minimum threshold force or above a maximum threshold force.

In some aspects, the processor may be configured to determine an amount of time the first force sensor senses the force.

In further aspects, the processor may be configured to determine whether an actuation of the first activation button is intentional or accidental by determining if the determined amount of time is below a minimum threshold amount of time or above a maximum threshold amount of time.

In other aspects, the processor may be configured to delay an amount of time between an actuation of the first activation button and a performance of an associated function of the surgical instrument by at least the minimum threshold amount of time.

In aspects, the maximum threshold force may be greater than the minimum threshold force.

In other aspects, the minimum and maximum threshold forces may be equal.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a robotic surgical system including a surgical robotic arm in accordance with the present disclosure; and

FIG. 2 is a side, perspective view of the surgical robotic arm of FIG. 1 coupled to a surgical instrument and a base.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to that portion of the robotic surgical system or component thereof, that is closer to a patient, while the term “proximal” refers to that portion of the robotic surgical system or component thereof, that is further from the patient.

As will be described in detail below, provided is a surgical robotic arm including a plurality of elongate members or links that are interconnected with one another and rotatable relative to one another. The robotic arm has one or more force sensors configured to sense a force associate with an actuation of one or more activation buttons. The activation buttons may be attached to the robotic arm, an instrument drive unit/surgical instrument slidably supported on the robotic arm, or a base on which the robotic arm is swivelably supported. A processor is provided that is adapted to determine, based on the force sensed by the one or more force sensors, whether an actuation of the one or more activation buttons was intentional (e.g., an intentional actuation by a clinician) or inadvertent (e.g., an accidental contact between the activation button and an object in an operating room).

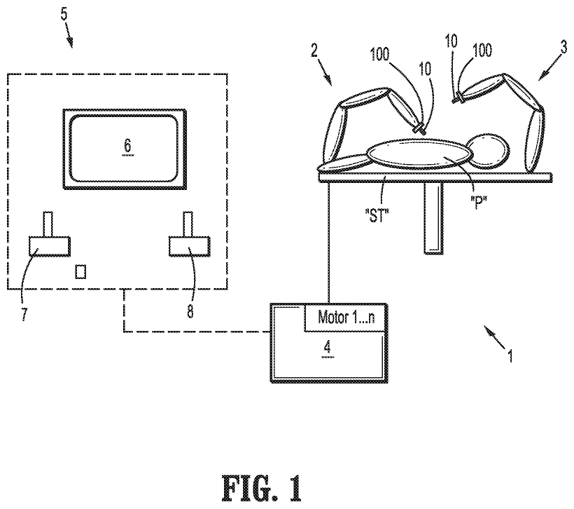

Referring initially to FIG. 1, a surgical system, such as, for example, a robotic surgical system 1, generally includes a plurality of surgical robotic arms 2, 3 having an instrument drive unit 100 and an electromechanical instrument 10 removably attached thereto; a control device 4; and an operating console 5 coupled with control device 4.

Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art. Each of the robotic arms 2, 3 may be composed of a plurality of members, which are connected through joints, as will be described in greater detail below. Robotic arms 2, 3 may be driven by electric drives (not shown) that are connected to control device 4. Control device 4 (e.g., a computer) is set up to activate the drives, in particular by means of a computer program, in such a way that robotic arms 2, 3, the attached instrument drive units 100, and thus electromechanical instrument 10 execute a desired movement according to a movement defined by means of manual input devices 7, 8. Control device 4 may also be set up in such a way that it regulates the movement of robotic arms 2, 3 and/or of the drives.

Robotic surgical system 1 is configured for use on a patient “P” lying on a surgical table “ST” to be treated in a minimally invasive manner by means of a surgical instrument, e.g., electromechanical instrument 10. Robotic surgical system 1 may also include more than two robotic arms 2, 3, the additional robotic arms likewise being connected to control device 4 and being telemanipulatable by means of operating console 5. A surgical instrument, for example, electromechanical surgical instrument 10, may also be attached to the additional robotic arm.

Control device 4 may control a plurality of motors, e.g., motors (Motor 1 . . . n), with each motor configured to drive movement of robotic arms 2, 3 in a plurality of directions. Further, control device 4 may control a motor, such as, for example, a hollow core motor, configured to drive a relative rotation of elongate members of surgical robotic arm 2.

For a detailed description of the construction and operation of a robotic surgical system, reference may be made to U.S. Pat. No. 8,828,023, entitled “Medical Workstation,” the entire contents of which are incorporated by reference herein.

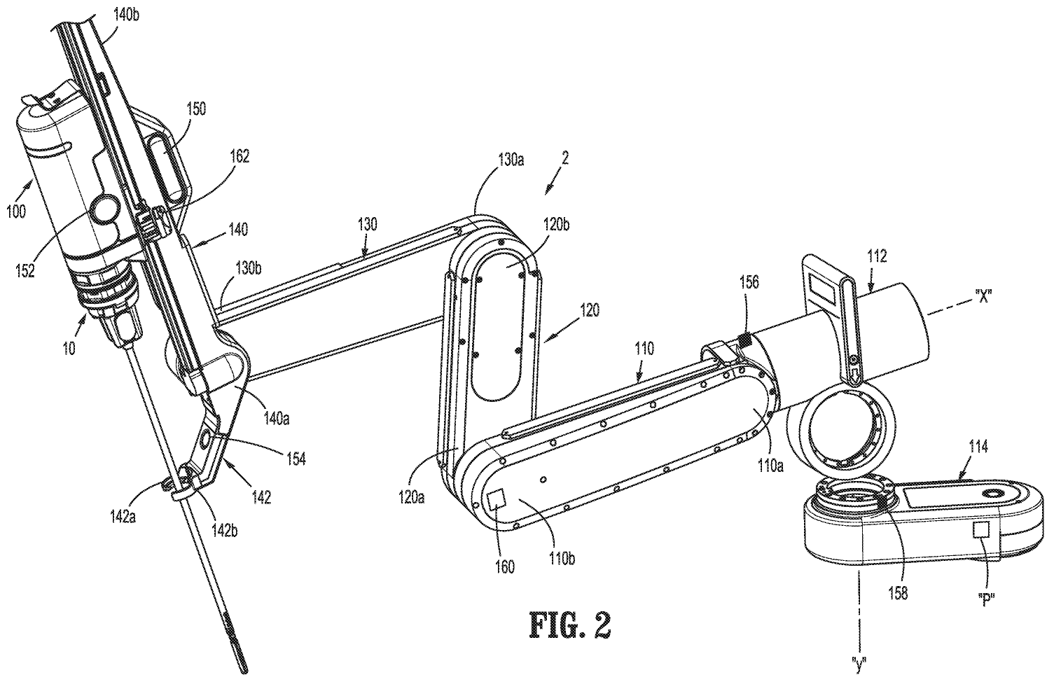

With reference to FIG. 2, the surgical robotic arm 2 is configured to support the instrument drive unit 100 thereon and to selectively move the instrument drive unit 100 and the attached surgical instrument 10 in a plurality of orientations relative to a small incision in a patient (e.g., a remote center of motion) while maintaining surgical instrument 10 within the small incision. The robotic arm 2 includes a plurality of elongate members or links 110, 120, 130 pivotably connected to one another to provide varying degrees of freedom to the robotic arm 2. In particular, the robotic arm 2 includes a first elongate member 110, a second elongate member 120, a third elongate member 130, and a fourth elongate member or rail 140.

The first elongate member 110 has a first end 110a and a second end 110b. The first end 110a is rotatably coupled to a connector 112. The connector 112 is rotatably coupled to a fixed surface or base 114, for example, a surgical cart, a surgical table, stanchion, gantry, operating room wall or ceiling, or other surface present in the operating room. The first end 110a of the first elongate member 110 is rotatable relative to the connector 112 about a longitudinal axis “X,” and the connector 112 is swivelable (or pivotable, rotatable, or articulatable) relative to the base 114 about a swivel axis “Y” that is perpendicular relative to the longitudinal axis “X” of the connector 112. The second end 110b of the first elongate member 110 is coupled to a first end of 120a of the second elongate member 120 and configured to rotate relative to the first elongate member 110 about a pivot axis defined through the second end 110b of the first elongate member 110 and the first end 120a of the second elongate member 120. The third elongate member 130 includes a first end 130a rotatably coupled to the second end 120b of the second elongate member 120, and a second end 130b.

It is contemplated that the robotic arm 2 has a plurality of motors, such as, for example, hollow core or pancake motors (not shown) disposed at each of the joints for driving the relative rotation of the elongate members 110, 120, 130. A motor (not shown) may also be provided in the connector 112 for driving a rotation of the first elongate member 110 relative to the connector 112, and a motor (not shown) may be provided in the base 114 for driving the swivel motion of the connector 112, along with the attached robotic arm 2, relative to the base 114.

The robotic arm 2 further includes an instrument carrier, slide or rail 140. The rail 140 has a first end 140a rotatably coupled to the second end 130b of the third elongate member 130. The rail 140 defines a longitudinal axis along which the surgical instrument 10 is slidable. The surgical instrument 10 is configured to rail along the longitudinal axis defined by the rail 140 upon a selective actuation by motor(s) (not shown) supported on the rail 140 or motors (1 . . . n) of the control device 4 (FIG. 1). As such, the surgical instrument 10 can be moved to a selected position along the rail 140.

The surgical robotic system 1 further includes a plurality of activation buttons 150, 152, 154 for actuating a particular function of the surgical instrument 10 or any other suitable component of the surgical robotic system 1, a plurality of sensors 156, 158, 160, 162, such as, for example, force sensors configured to sense a force associated with a physical actuation of one or more of the activation buttons 150, 152, 154, and a processor “P” in communication with the sensors 156, 158, 160, 162 and/or the buttons 150, 152, 154.

A first activation button 150 may be attached to the rail 140 and configured to effect a selective locking of the instrument drive unit 100 in position relative to the rail 140. It is contemplated that an actuation of the first activation button 150 may perform other functions, such as, for example, rotate the robotic arm 2 about and relative to the longitudinal axis “X” defined by the connector 112. A second activation button 152 may be attached to the instrument drive unit 100 and configured to effect a selective locking of the surgical instrument 10 relative to the instrument drive unit 100. It is contemplated that an actuation of the second activation button 152 may perform other functions, such as, for example, rotate the surgical instrument 10 relative to the instrument drive unit 100 about the longitudinal axis of the surgical instrument 10. A third activation button 154 may be attached to the first end 140a of the rail 140 and configured to effect a selective locking of a pair of clamp arms 142a, 142b of a trocar mount 142 supported at the end of the rail 140. It is contemplated that an actuation of the third activation button 154 may perform other functions, such as, for example, rotate the rail 140 about a pivot axis extending perpendicularly through the second end 130b of the third elongate member 130 and the first end 140a of the rail 140.

In some aspects, the activation buttons 150, 152, 154 may be located at any other suitable location of the surgical robotic system 1. In further aspects, the surgical robotic system 1 may include more or less than three activation buttons.

The force sensors 156, 158, 160, 162 are attached to various locations of the surgical robotic system 1. For example, a first force sensor 156 may be attached at the joint connecting the connector 112 and the first end 110a of the first elongate member 110 of the robotic arm 2, such that the first force sensor 156 senses the force associated with a rotation of the robotic arm 2 relative to the connector 112 about the longitudinal axis “X.” Accordingly, the first force sensor 156 may sense the force associated with an actuation of the first activation button 150 and/or the second activation button 152. A second force sensor 158 may be attached to the joint connecting the base 114 and the connector 112, such that the second force sensor 158 senses the force associated with a rotation of the robotic arm 2/connector 112 relative to the base 114 and about the pivot axis “Y.” Accordingly, the second force sensor 158 may sense the force associated with an actuation of the first activation button 150 and/or the second activation button 152. A third force sensor 160 may be attached to the joint connecting the second end 110b of the first elongate member 110 and the first end 120a of the second elongate member 120, such that the third force sensor 160 senses the force associated with a rotation of the second elongate member 120 relative to the first elongate member 110. Accordingly, the third force sensor 160 may sense the force associated with an actuation of the third activation button 154. The force sensors may be force-sensing resistors, force and/or pressure sensing MEMS devices, torque sensors, strain gauges, or the like.

The surgical robotic system 1 may include more than three force sensors. For example, the surgical robotic system 1 include a fourth force sensor 162 attached to the interface between the instrument drive unit 100 and the rail 140 to sense a force exerted on the instrument drive unit 100 intended to move the instrument drive unit 100 and the attached surgical instrument 10 along the rail 140.

The processor “P” may be incorporated into the control device 4 (FIG. 1) or be disposed at any other suitable location of the surgical robotic system 1, such as, for example, the base 114 or the robotic arm 2. The processor “P” may be operably connected to a memory, which may include transitory type memory (e.g., RAM) and/or non-transitory type memory (e.g., flash media, disk media, etc.). The processor “P” includes an output port that is operably connected to a power source allowing the processor to control the output of the power source according to either open and/or closed control loop schemes. A closed loop control scheme is a feedback control loop, in which the force sensors 156, 158, 160, 162 measure a force and provide feedback to the processor “P.” The processor “P” is configured to then signal the power source, which adjusts the power supplied to the surgical robotic system 1. Those skilled in the art will appreciate that the processor “P” may be substituted by using any logic processor (e.g., control circuit) adapted to perform the calculations and/or set of instructions described herein including, but not limited to, field programmable gate arrays, digital signal processor, and combinations thereof. The processor “P” is capable of executing software instructions for processing the data received by the force sensors 156, 158, 160, 162, and for outputting control signals to the power supply, accordingly. The software instructions, which are executable by the processor “P”, are stored in the memory.

The processor “P” is configured to determine whether an actuation of any of the activation buttons 150, 152, 154 is an intentional act by a clinician or accidental based on data received from one or more of the force sensors 156, 158, 160, 162. The processor “P” determines whether an actuation of any of the activation buttons 150, 152, 154 was an intentional act by a clinician or accidental by determining if the force sensed by either of the force sensors 156, 158, 160, 162 is below a minimum threshold force or above a maximum threshold force. The minimum threshold force is the minimum amount of force required to depress one of the activation buttons 150, 152, 154, whereas the maximum threshold force is one that exceeds the force typically used by a clinician to depress one of the activation buttons 150, 152, 154. If the determined force is below the threshold force or above the threshold force, the processor “P” determines that the actuation of the selected activation button 150, 152, or 154 is accidental and will not allow for the actuation of the activation button 150, 152, or 154 to bring about the associated function. In embodiments, the minimum and maximum threshold forces may be equivalent. In other embodiments, the minimum threshold force is less than the maximum threshold force.

The processor “P” may be further configured to determine the amount of time one or more of the force sensors 156, 158, 160, 162 senses the force associated with the actuation of one of the activation buttons 150, 152, 154. If the determined amount of time is below a minimum threshold amount of time or exceeds a maximum threshold amount of time, the processor “P” determines that actuation of the selected activation button 150, 152, 154 was accidental (e.g., the robotic arm 2 bumps into an object in the operating room or the activation button 150, 152, or 154 is jammed) and will not allow for the actuation of the activation button 150, 152, 154 to effect the associated function. As such, the processor “P” delays the amount of time between actuation of one of the activation buttons 150, 152, 154 and a performance of the associated function by at least the minimum threshold amount of time. In embodiments, the minimum threshold amount of time and the maximum threshold amount of time are equivalent. In other embodiments, the minimum threshold amount of time is less than the maximum threshold amount of time.

If the determined force is between the minimum threshold force and the maximum threshold force and the determined amount of time is between the minimum threshold time and the maximum threshold time, the processor “P” is configured to permit the actuation of the activation button 150, 152, 154 to effect the associated function.

The memory may have stored therein the location of the sensors 156, 158, 160, 162, such that the processor “P” may back-calculate loads applied to the system based on the location of the sensors 156, 158, 160, 162 and the determined forces. For example, if one of the force sensors on the robotic arm 2, such as force sensor 160, measures a force of 10N and the force sensor 162 on the slide 140 measures a force of 20N, the processor “P” may be configured to extrapolate that there is an additional load somewhere else on the system. The processor “P” may be further configured to use geometry and configuration to determine moment loads on the system when a person leans on the system.

In some embodiments, the surgical robotic system 1 may be equipped with features that alert the clinician when an actuation of an activation button 150, 152, 154 is intentional or accidental. For example, the surgical robotic system 1 may include lights (e.g., LEDs) on the robotic arm 2 that turn on or off depending on whether an actuation of an activation button 150, 152, 154 is found to be intentional or accidental. The surgical robotic system 1 may also be equipped with speakers that emit a sound depending on whether an actuation of an activation button 150, 152, 154 is found to be intentional or accidental.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical robotic system, comprising:
a robotic arm including a plurality of elongate members rotationally coupled to one another, at least one of the plurality of elongate members configured to have a surgical instrument attached thereto;
a first activation button coupled to at least one of the plurality of elongate members and configured to actuate a function;
a first force sensor coupled to at least one of the plurality of elongate members and configured to sense a force associated with an actuation of the first activation button; and
a processor in communication with the first force sensor, wherein the processor is configured to determine whether an actuation of the first activation button is intentional or accidental based on data received from the first force sensor.

2. The surgical robotic system according to claim 1, wherein the plurality of elongate members includes a rail having the surgical instrument slidably coupled thereto, the first activation button being disposed with the rail.

3. The surgical robotic system according to claim 2, further comprising a second activation button attached to an instrument drive unit of the surgical robotic system, the instrument drive unit configured to drive an operation of the surgical instrument.

4. The surgical robotic system according to claim 3, further comprising a second force sensor configured to sense a force associated with an actuation of the second activation button.

5. The surgical robotic system according to claim 1, wherein the plurality of elongate members includes:
a first elongate member having a first end and a second end;
a second elongate member having a first end rotatably connected to the second end of the first elongate member, and a second end;
a third elongate member having a first end rotatably connected to the second end of the second elongate member, and a second end; and
a rail rotatably coupled to the second end of the third elongate member and having the surgical instrument slidably coupled thereto, the first activation button being disposed with the rail.

6. The surgical robotic system according to claim 5, further comprising a base, wherein the first end of the first elongate member is rotatably coupled to the base.

7. The surgical robotic system according to claim 5, further comprising:
a second activation button attached to the surgical instrument, an instrument drive unit, or the robotic arm and configured to actuate a function; and
a second force sensor disposed with the base and configured to sense a force associated with an actuation of the second activation button.

8. The surgical robotic system according to claim 6, wherein the first elongate member is configured to rotate relative to the base about a first pivot axis, and wherein the second elongate member is configured to rotate relative to the first elongate member about a second pivot axis, extending perpendicularly relative to the first pivot axis.

9. The surgical robotic system according to claim 8, wherein the surgical instrument is configured to move along a longitudinal axis defined by the rail.

10. The surgical robotic system according to claim 1, wherein the processor is configured to determine whether an actuation of the first activation button is intentional or accidental by determining if the force sensed by the first force sensor is below a minimum threshold force or above a maximum threshold force.

11. The surgical robotic system according to claim 10, wherein the processor is configured to determine an amount of time the first force sensor senses the force.

12. The surgical robotic system according to claim 11, wherein the processor is configured to determine whether an actuation of the first activation button is intentional or accidental by determining if the determined amount of time is below a minimum threshold amount of time or above a maximum threshold amount of time.

13. The surgical robotic system according to claim 12, wherein the processor is configured to delay an amount of time between an actuation of the first activation button and a performance of an associated function of the surgical instrument by at least the minimum threshold amount of time.

14. The surgical robotic system according to claim 10, wherein the maximum threshold force is greater than the minimum threshold force.

15. The surgical robotic system according to claim 10, wherein the minimum and maximum threshold forces are equal.
